(19) United States
(12) Patent Application Publication
(10) Pub. No.: US 2019/0282829 A1
(43) Pub. Date: Sep. 19, 2019

(54) METHODS AND APPARATUS FOR TREATMENT OF DISORDERS

(71) Applicants: THE GENERAL HOSPITAL CORPORATION, Boston, MA (US); MASSACHUSETTS EYE AND EAR INFIRMARY, Boston, MA (US)

(72) Inventors: Paolo Cassano, Lexington, MA (US); Husam Katnani, Boston, MA (US); Michael Hamblin, Boston, MA (US); Benjamine Bleier, Boston, MA (US)

(21) Appl. No.: 16/300,658

(22) PCT Filed: May 15, 2017
(86) PCT No.: PCT/US2017/032760

§ 371 Date: Nov. 12, 2018

Related U.S. Application Data
(60) Provisional application No. 62/336,221, filed on May 13, 2016.

(51) Int. Cl. A61N 5/06; A61B 5/16

(52) U.S. Cl. CPC A61N 5/0618; A61N 5/0603; A61N 5/0622; A61B 5/165; A61N 2005/0662; A61N 2005/0626; A61N 2005/0651; A61N 2005/0659; A61N 2005/0607

(57) ABSTRACT

A method is provided for controlling a device configured for treating a disorder of a subject. The method comprises providing power to an implantable device configured to be located within a submucosa of a nasal cavity of the subject to cause the implantable device to emit near-infrared light and red light directed to at least one of regions of an ocular structure, regions of a cerebrum, cerebral nerves, and cerebrospinal fluid, regions of a vascular system, and regions of a lymphatic system of the subject. The device is configured to be implanted in a position to deliver the near-infrared light and the red light to the at least one of the regions of the ocular structure, the regions of the cerebrum, cerebral nerves, and cerebrospinal fluid, regions of the vascular system, and the regions of the lymphatic system in a dosimetry and duration sufficient to treat the disorder.

### CROSS-REFERENCE TO RELATED APPLICATIONS

This application is based on, claims priority to, and incorporates herein by reference in its entirety U.S. Provisional Application Ser. No. 62/336,221, entitled “METHODS AND APPARATUS FOR TREATMENT OF BRAIN DISORDERS,” and filed Mar. 13, 2016.

### STATEMENT REGARDING FEDERALLY SPONSORED RESEARCH

N/A

### BACKGROUND

More than 23 million Americans suffer from Major Depressive Disorder (MDD) every year. Further, MDD is associated with $106-$118 billion/year of total societal costs in the United States. Depressive disorders are the leading cause of years lost to disability worldwide. Even when properly treated with antidepressants, almost 2 million subjects with MDD fail to achieve remission from depression, with persistence of suffering. For these individuals, electro-convulsive therapy (ECT) is the main recourse. Even then, approximately 50% of depressed subjects fail to achieve adequate response with ECT. Options for treatment resistant subjects are limited. Transcranial magnetic stimulation has been shown not to have effective long-term efficacy, with over 60% of subjects failing to remit or subsequently relapsing. Deep brain stimulation, which involves implantation of brain leads, has not shown significant advantage over sham treatment. Recent treatments, such as intravenous ketamine, lack enduring antidepressant efficacy. Consequently, there is a serious unmet medical need for new treatments to benefit those severely resistant to available treatments.

### SUMMARY

The present disclosure overcomes the aforementioned drawbacks by providing a method of treating a subject with an implantable LED device, which efficiently delivers near-infrared light (NIR) and red light to the brain from an intranasal site.

In accordance with one aspect of the disclosure, a method is provided for controlling a device configured for treating a disorder of a subject. The method comprises providing power to an implantable device configured to be located within a submucosa of a nasal cavity of the subject to cause the implantable device to emit near-infrared light and red light directed to at least one of regions of an ocular structure, regions of a cerebrum, cerebral nerves, and cerebrospinal fluid, regions of a vascular system, and regions of a lymphatic system of the subject, the near-infrared light having a wavelength of 750 nm to 1200 nm, the red light having a wavelength of between 600 nm and 749 nm. The device is configured to be implanted in a position to deliver the near-infrared light and the red light to the at least one of the regions of the ocular structure, the regions of the cerebrum, cerebral nerves, and cerebrospinal fluid, regions of the vascular system, and the regions of the lymphatic system in a dosimetry and duration sufficient to treat the disorder.

In some aspects, the device can be configured to deliver the near-infrared light and the red light to the regions of the cerebrum through at least a portion of a cribriform plate of the subject. The implantable device can be sized to be located within the submucosa of the nasal cavity, in close proximity to the cribriform plate of the subject, and below a cranial base of the subject. The implantable device can be sized to be located between 0.1 cm and 4 cm from the cribriform plate of the subject.

In some other aspects, providing power to the implantable device can include wirelessly delivering power to the implantable device using a remote generator.

In yet some other aspects, providing power to the implantable device can include delivering the power wirelessly form a wearable remote generator configured to be worn by the subject.

In still some other aspects, the method further comprises controlling the implantable device to deliver the near-infrared light and the red light simultaneously.

In some other aspects, the method further comprises delivering the near-infrared light at one of a wavelength of about 825 nm, a wavelength of about 850 nm, or a wavelength of about 808 nm to about 830 nm.

In yet some other aspects, the method further comprises delivering the red light at one of a wavelength of about 620 nm to about 633 nm or a wavelength of about 633 nm.

In still some other aspects, the red light can comprise about 1% to about 50% of a total light delivered.

In some other aspects, the method further comprises controlling the implantable device to deliver the near-infrared light and red light together in a series of alternating pulses, wherein near-infrared light is at a wavelength of about 795 nm to about 830 nm and red light is at a wavelength of about 650 nm to about 720 nm in a pulse that alternates with a next pulse, wherein near-infrared light is at a wavelength of about 721 nm to about 794 nm and red light is at a wavelength of about 600 nm to about 649 nm.

In yet some other aspects, the method further comprises controlling the implantable device to deliver the near-infrared light and red light in a series of alternating pulses, wherein near-infrared light is at a wavelength of about 760 nm to about 830 nm and red light is at a wavelength of about 620 nm to about 680 nm.

In still some other aspects, the method further comprises controlling the implantable device to deliver a duration of administration of near-infrared light and red light of about 1 minute to about 120 minutes per day.

In some other aspects, the method further comprises controlling the implantable device to deliver a duration of administration of near-infrared light and red light of about 1 minute to 120 minutes once, twice or three times per week or daily or 20 times per day.

In yet some other aspects, the regions of the cerebrum can be at least one of the ventromedial prefrontal cortex (vmPFC), subgenual anterior cingulate cortex (ACC) and the olfactory bulb.

In still some other aspects, the disorder can be at least one of a depressive disorder, an anxiety disorder, a trauma- and stressor-related disorder, a disorder manifesting with suicidal ideation or just suicidal ideation, a nicotine addiction disorder, an alcohol use disorder, a substance use disorder, a sexual dysfunction disorder, a neurocognitive disorder, an attention deficit and hyperactivity disorder, a sleep-wake disorder, a disorder associated with chronic fatigue syndrome, a disorder associated with fibromyalgia, a somatic symptom disorder, an eating disorder, a psychotic disorder, an obsessive-compulsive disorder, a cluster-B personality disorder, a disruptive, impulse-control, and conduct disorder, and an otorhinolaryngology disorder.

In some other aspects, the subject can have been diagnosed with treatment resistant depression.

In yet some other aspects, the near-infrared light can comprise about 50% to about 99% of a total light delivered. The near-infrared light can comprise about 75% of the total light delivered.

In still some other aspects, controlling the implantable device can include causing the implantable device to deliver the near-infrared light and red light to achieve between about 5 mW/cm² to about 700 mW/cm² irradiance, between about 1 J/cm² to about 300 J/cm² fluence, with one of continuous light and 1 Hz to about 100 Hz pulses of near-infrared light and red light. The irradiance can be between about 22 mW/cm² to about 33 mW/cm², the fluence can be between about 9.56 J/cm² to about 12 J/cm², and the dosimetry of near-infrared light and red light administered to the subject can comprise about 10 Hz pulses of near-infrared light and red light.

In accordance with another aspect of the disclosure, a device is provided that is configured for treating a disorder of a subject. The device comprises a power source, a light source, and a housing. The light source is configured to receive power from the power source to cause the light source to emit near-infrared light and red light directed, wherein the near-infrared light has a wavelength of 750 nm to 1200 nm and the red light has a wavelength of between 600 nm and 749 nm. The housing is configured to be located within a submucosa of a nasal cavity of the subject to position the light source to deliver the near-infrared light and the red light toward at least one of regions of an ocular structure, regions of a cerebrum, cerebral nerves, and cerebrospinal fluid, regions of a vascular system, and regions of a lymphatic system of the subject in a dosimetry and duration sufficient to treat the disorder.

In some other aspects, the light source can be configured to deliver the near-infrared light and the red light to the regions of the cerebrum through at least a portion of a cribriform plate of the subject. The housing can be sized to be located within the submucosa of the nasal cavity, in close proximity to the cribriform plate of the subject, and below a cranial base of the subject. The power source can be configured to wirelessly deliver the power to the light source.

Other features and advantages of the invention will be apparent from the Detailed Description, and from the claims. Thus, other aspects of the invention are described in the following disclosure and are within the ambit of the invention.

The foregoing and other aspects and advantages of the invention will appear from the following description. In the description, reference is made to the accompanying drawings that form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIG. 1 is a schematic diagram depicting a system for deep intranasal light (DIL) delivery.

FIG. 2A is a front elevational view of an exemplary handheld device for use with the system of FIG. 1, shown with LED components retracted into a guide shaft.

FIG. 2B is a front elevational view of the exemplary handheld device of FIG. 2A, shown with the LED components actuated partially out of the guide shaft.

FIG. 3 is a schematic diagram depicting a system for deep intranasal light (DIL) delivery from an implantable device.

FIG. 4 is a schematic diagram depicting a telemetry link for use with the system of FIG. 3.

FIG. 5 is a cross-sectional view of a nasal region of a subject, illustrating where the implantable device of FIG. 3 can be located within the submucosal tissue.

FIG. 6A is a side cross-sectional view of a model subject's skull, showing the penetration of near infrared and red light from a deep intranasal light source.

FIG. 6B is a rear cross-sectional view of a model subject's skull, showing the penetration of near infrared and red light from a deep intranasal light source.

FIG. 7 is a side cross-sectional view of a model subject's skull, showing the penetration of near infrared and red light from a superficial light source.

FIG. 8 is a rear cross-sectional view of a model subject's skull, showing the location of the deep intranasal light source of FIGS. 6A and 6B within the nasal region of the model subject.

FIG. 9 is a rear cross-sectional view of a model subject's skull, showing the penetration of near infrared and red light from another deep intranasal light source.

FIG. 10 is a rear cross-sectional view of a model subject's skull, showing the location of the deep intranasal light source of FIG. 9 within the nasal region of the model subject.

FIG. 11A is a chart illustrating the mean HAM-D₁₇ total scores over a course of a study for a first transcranial photobiomodulation group.

FIG. 11B is a chart illustrating the mean HAM-D₁₇ total scores over a course of a study for a second transcranial photobiomodulation group.

FIG. 12 is a schematic view of a controller configured for use with any of the systems described herein.

The following Detailed Description, given by way of example, but not intended to limit the invention to specific embodiments described, may be understood in conjunction with the accompanying figures, incorporated herein by reference.

### DETAILED DESCRIPTION

Unless otherwise defined, all technical and scientific terms used herein have the same meaning as commonly understood by one of ordinary skill in the art to which this invention belongs. In case of conflict, in the present application, included definitions will control.

A “subject,” as used herein, is a vertebrate, including any member of the class Mammalia, including humans, domestic and farm animals, and zoo, sports or pet animals, such as mouse, rabbit, pig, sheep, goat, cattle and higher primates.

As used herein, the terms “treat,” “treating,” “treatment,” and the like refer to reducing or ameliorating a brain disorder and/or symptoms associated therewith. It will be appreciated that, although not precluded, treating a brain disorder or condition does not require that the disorder, condition, or symptoms associated therewith be completely eliminated.

Unless specifically stated or clear from context, as used herein, the term “about” is understood as within a range of normal tolerance in the art, for example within 2 standard deviations of the mean. “About” is understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

Infrared (IR) light is ubiquitously present to most life on the earth. Of the total amount of solar energy reaching the human skin, 54% is IR and 30% is IR type A—near-infrared—(NIR; with a wavelength range of 760 to 1440 nm) which penetrates through the human skin and reaches deeply into tissue, depending on wavelength and energy. NIR can be used to treat a variety of conditions such as muscle pain, wounds, neuropathic pain, and headaches. NIR can also be used for wellness and lifestyle purposes such as for cosmetic improvement in peri-orbital wrinkles. NIR can, in some instances, be used for transcranial phototherapy to treat various brain disorders. For example, NIR can be used to treat a subject who has an acute stroke. Numerous preclinical animal studies suggested that the application of NIR laser (810 nm) to the head at various times (hours) after induction of an acute stroke had beneficial effects on subsequent neurological performance and reduced lesion size.

To treat various disorders, NIR radiation can target various cellular structures. Specifically, the NIR photons can be absorbed by cytochrome c oxidase in the mitochondrial respiratory chain. This mitochondrial stimulation increases production of adenosine triphosphate (ATP), but also activates signaling pathways by a brief burst of reactive oxygen series (ROS). This signaling activates antioxidant defenses reducing overall oxidative stress. Proinflammatory cytokines and neuroinflammation are reduced. Neurotrophins such as brain-derived neurotrophic factor are upregulated, which in turn activates synaptogenesis (formation of new connections between existing neurons) and neurogenesis (formation of new neurons from neural stem cells) throughout treated areas in the brain. Evidence has also shown anti-inflammatory and anti-apoptotic effects in the brain stimulated by this approach.

Specific parts of the brain govern specific functions of the mind and body. For example, the diencephalon (roughly around the mid-brain) is the seat of some of the most essential survival functions, and holds some keys to the physical well-being of the person. Among the sub-regions here, the hypothalamus is the control center for many autonomic functions. It is connected with structures of the endocrine and nervous systems to support its vital role in maintaining homeostasis throughout the body. It is part of the limbic system that influences various emotional and pleasure responses, storing memories, regulating hormones, sensory perception, motor function, and olfaction. The other components of the limbic system are the amygdala, cingulated gyrus, hippocampus, olfactory cortex and the thalamus.

Whilst the mid-brain area could be a primary target for NIR treatment, the divergent light rays can also illuminate other parts of the brain (or other organs generally) to achieve a wider spread benefit. In some instances, the substantia nigra (its dysfunction lead to Parkinson's disease) located at the bottom of the mid-brain area, or another location in the prefrontal cortex, could be targeted to improve higher order cognitive functions.

When selected portions of the brain are receiving light treatment, the effects can further be rapidly distributed throughout the brain through the neural network. The key to the response of the brain lies in the presence of a photoacceptor respiratory enzyme in all cellular mitochondria called cytochrome oxidase. It represents the best known intraneural marker of metabolic activity and is tightly coupled with free radical metabolism, cell death pathway, and glutamatergic (a neurotransmitter related) activation, important for learning and memory.

Photoacceptors, unlike photoreceptors found inside the eyes, do not process light, but are part of metabolic pathways. They are sensitive to light in the visible red and near-infrared parts of the spectrum, and convert the absorbed light into cellular energy ATP. When light with these wavelengths at low energy hits the cells (including nerve cells), it modulates the cells into metabolism (photobiomodulation) by regulating mitochondrial function, intraneuronal signaling systems, and redox states. With the brain affecting virtually all functions of the body, the impact of exposing neurons to light (photoneurobiomodulation) could consequently affect the entire well-being of the human being.

The sensitivity of cytochrome oxidase to red and near infrared red light can be explained by the role of a chromophore in the protein structure. This chromophore is an organic cofactor that is present in all photoreceptors, such as those in the eyes that give us the perception of colors. These chromophores will absorb particular wavelengths and reject the others, and those in the cytochrome accept red and infrared red light. These facts express the potential impact of light that could be correctly directed to the various parts of the brain, resulting in both therapy for, and prophylaxis against various disorders, such as, for example, Major Depressive Disorder (MDD).

Animal research has shown that PBM stimulates neurogenesis and protects against cell death. Data suggest that red light, close to the NIR spectrum (670 nm), protects the viability of cell culture after oxidative stress, as indicated by mitochondria membrane potentials. NIR also stimulates neurite outgrowth mediated by nerve growth factor, and this effect could also have positive implications for axonal protection. Neuroprotective effects of red light (670 nm) were documented in in vivo models of mitochondrial optic neuropathy. Red light close to NIR spectrum (670 nm) has also been shown to protect neuronal cells against cyanide. In animal models of TBI, NIR (810 nm) appears to be an effective treatment and improves neurogenesis and synaptogenesis, via increase of brain-derived neurotrophic factor (BDNF). In addition, NIR improves memory performance in middle-aged mice.

In summary, PBM increases neurotrophins, neurogenesis, synaptogenesis, and ATP, while it reduces inflammation, apoptosis, and oxidative stress. Through these mechanisms, PBM has the potential to be an effective treatment for MDD and comorbid disorders.

Multiple studies have reported regional and global hypometabolism in MDD, which could be related (either causally or consequentially) to the neurobiology of mood disorders. Positron emission tomography studies have shown abnormalities in glucose consumption rates and in blood flow in several brain regions of subjects with major depression. Moreover, metabolic abnormalities in the anterior cingulate, the amygdala-hippocampus complex, the dorsolateral prefrontal cortex (DLPFC), and inferior parietal cortex seem to improve after antidepressant treatment or after recovery.

In experimental and animal models, PBM (NIR and red light) noninvasively delivers energy to the cytochrome c oxidase and by stimulating the mitochondrial respiratory chain leads to increased ATP production. A study of the effects of NIR on subjects with MDD found that a single session of NIR led to a marginally significant increase in regional cerebral blood flow. Whether the observed changes in cerebral blood flow resulted from fundamental changes in neuronal metabolism or changes in vascular tone remain to be clarified. Given the correlation of both hypometabolism and abnormal cerebral blood flow with MDD, the beneficial effect of NIR on brain metabolism is one potential mechanism for its antidepressant effect.

Further, NIR light and red light (600 to 1600 nm) decreased synovial IL-6 gene expression (decreased mRNA levels) in a rat model of rheumatoid arthritis. In another study, NIR (810 nm) used as a treatment for pain in subjects with rheumatoid arthritis decreased production of the following proinflammatory cytokines: TNF-α, IL-1β, and IL-8. Khuman et al. showed that transcranial NIR improved cognitive function and reduced neuroinflammation as measured by Iba1+ activated microglia in brain sections from mice that had suffered a TBI. Finally, NIR (970 nm) has been found to be an effective treatment for inflammatory-type acne.

Oxidative stress may additionally be an effective target for antidepressant treatments. However, successful treatments for MDD vary in regard to their protective effects against oxidative stress. Animal research suggests that PBM may have beneficial effects on oxidative stress. In a rat model of traumatized muscle, NIR (904 nm) blocked the release of harmful ROS and the activation of the transcription factor, nuclear factor κB (NF-κB), both induced by muscle trauma. Trauma activates NF-κB by destroying a specific protein inhibitor of NF-κB called IκB, and this destruction was inhibited by NIR light. Furthermore, NIR reduced the associated overexpression of the inducible form of nitric oxide synthase (iNOS) and reduced the production of collagen. This regulation of iNOS is important because excessive levels of iNOS can lead to formation of large amounts of NO that combine with superoxide radicals to form the damaging species peroxynitrite, and can interfere with the protective benefits of other forms of NO synthase. These findings suggest that NIR protects against oxidative stress induced by trauma. Finally, an in vitro study of the effects of red light and NIR (700 to 2000 nm) on human RBCs found that NIR significantly protected RBCs against oxidation

As such, transcranial photobiomodulation (t-PBM) with near-infrared radiation (NIR) has emerged as a potential antidepressant treatment in both animal models and human studies. t-PBM consists of delivering NIR and/or red light to the scalp (generally predetermined locations on the forehead) of the subject, which penetrates the skull and modulates function of the adjacent cortical areas of the brain. PBM with red light and/or NIR appears to increase brain metabolism (by activating the cytochrome C oxidase in the mitochondria), to increase neuroplasticity, and to modulate endogenous opioids, while decreasing inflammation and oxidative stress.

t-PBM penetrates deeply into the cerebral cortex, modulates cortical excitability, and improves cerebral perfusion and oxygenation. Studies have suggested that it can significantly improve cognition in healthy subjects, and in subjects with traumatic brain injury (TBI). The safety of t-PBM has been studied in a sample of acute 1,410 stroke subjects, with no significant differences in rates of adverse events between t-PBM and sham exposure. Uncontrolled studies suggest an antidepressant effect of t-PBM in subjects suffering from major depressive disorder (MDD).

For the transcranial treatment of major depressive disorder (MDD), both PBM LEDs and lasers have been experimentally tested. Certain forms of PBM treatment are also referred to as low-level light therapy (LLLT), since it utilizes light at a low power (0.1 to 0.5 W output at the source) to avoid any heating of tissue. The irradiance of the PBM medical devices (or power density) typically ranges from 1 to 10 times the NIR irradiance from sunlight on the skin (33.6 mW/cm2 at the zenith). However, most PBM medical devices only deliver light energy at one or two selected wavelengths, as opposed to the whole spectrum of IR that is contained in sunlight.

However, transcranial photobiomodulation is both time-consuming and expensive. As such, aspects of the present disclosure provide a new technique based on intranasal photobiomodulation: Deep intranasal light (DIL).

DIL is light in the NIR and red spectrum, delivered intranasally to the brain, for example, with an endonasal catheter or an implantable device, wherein the light is delivered through a base of the skull. In some instances, this light may be delivered at the level or in proximity of the cribriform plate onto any of the olfactory bulb, ventromedial prefrontal cortex, subgenual cingulate cortex, or any other portion of the brain, as necessary for a desired treatment. The olfactory bulb is the most accessible part of the limbic system and is connected to the amygdala, hippocampus, orbitofrontal, and insular cortex, all implicated in the genesis of emotions and in the pathogenesis of depression and anxiety.

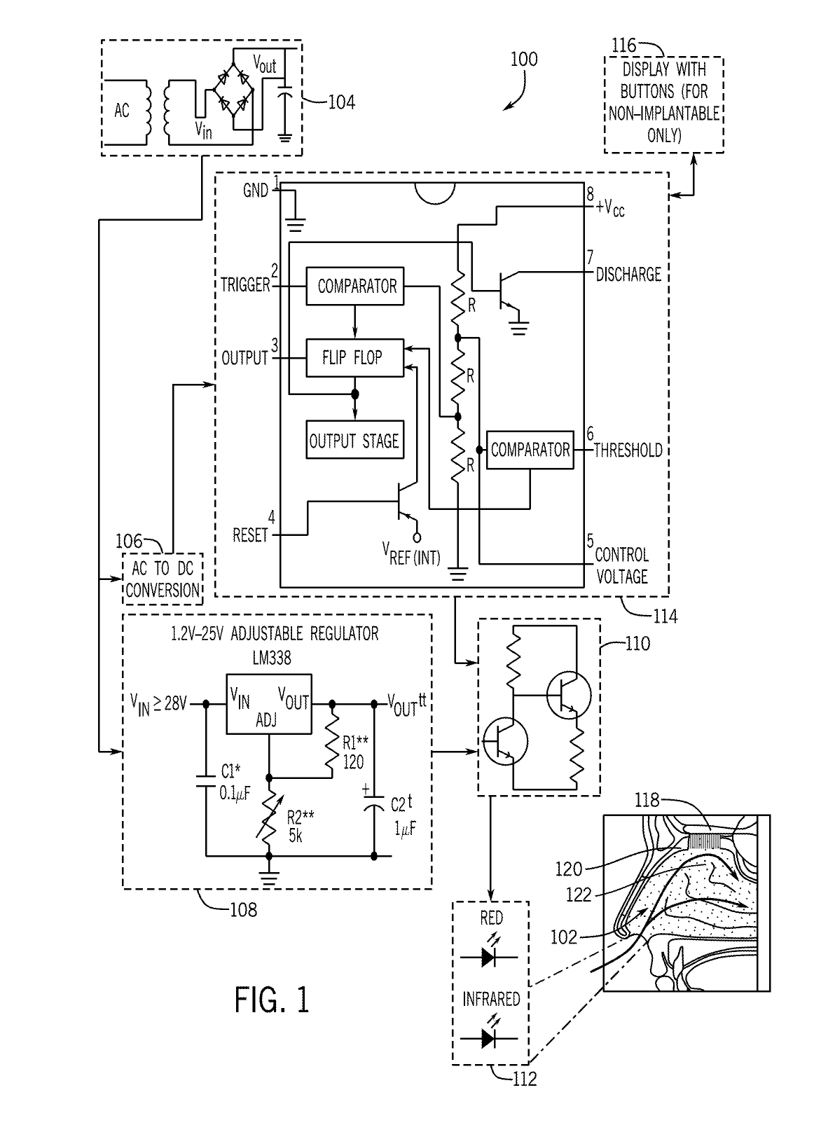

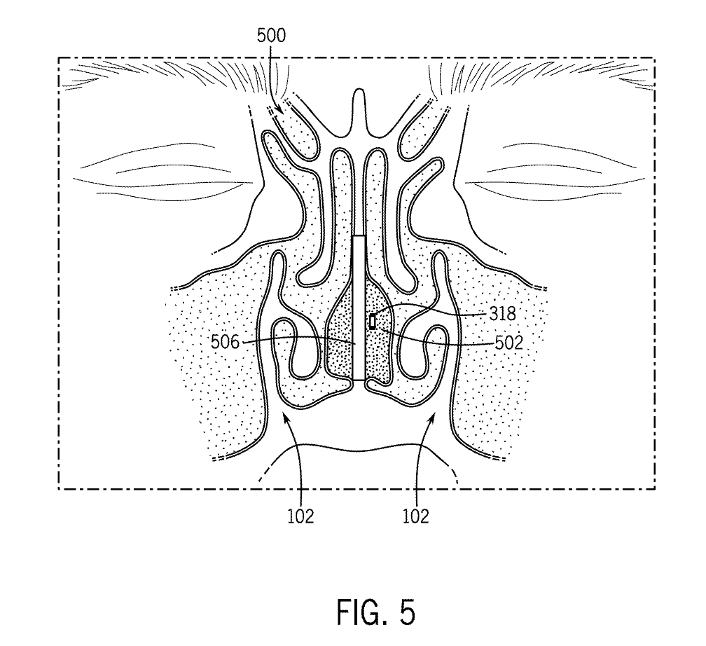

Referring to FIG. 1, one, non-limiting, example of a system 100 for deep intranasal light (DIL) delivery is illustrated. As alluded to above, the system 100 can be designed as a handheld device that can be extended into a nasal cavity 102 to a treatment location 122, or as an implantable device configured to be implanted in a submucosa tissue layer of the nasal cavity, as will be described below, with respect to FIG. 5. To this end, the system 100 can be designed to achieve direct access to the limbic system (e.g., olfactory bulb 118, ventromedial prefrontal cortex, subgenual anterior cingulate cortex) near the base of the skull, for example, through the cribriform plate 120. As will be described, the system 100 can be used to provide therapy and/or treatment related to various disorders, such as, for example, sexual dysfunction (e.g. decreased libido), depression, anxiety, cognitive impairment, and the like.

The system 100 includes a power source 104. In the illustrated, non-limiting example, the power source 104 may be designed to receive transmission-type AC power, such as from a wall outlet and, thus, includes a transformer and rectifying bridge. Alternatively, the power source 104 may include power storage components (i.e., batteries) or other DC sources. If a dedicated DC source is not included, such as is illustrated in FIG. 1, the system 100 may include a voltage regulator 106 that converts power into a lower DC source.

The system 100 may also include a gain controller 108 that allows the user to adjust the operational power and, for example, change light intensity. That is, the gain controller 108 is coupled to an LED driver 110 that can act as a switch depending on voltage input. Overall the LED driver 110 provides a constant output to LEDs 112 in order to maintain fidelity of light source and prevent LED damage.

A controller 114 is provided that is programmed to operate the system 100, for example, by controlling pulsing, frequencies, intensity modulation, and the like. Also, a display 116 or other user interface elements may be included to allow a user to interact with the controller 114.

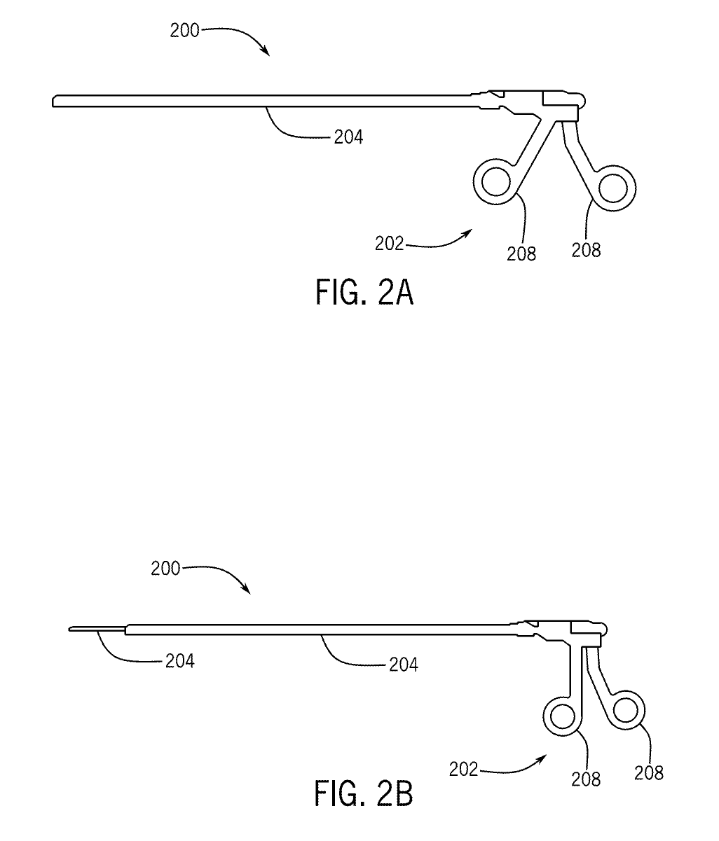

Referring now to FIGS. 2A and 2B, an exemplary handheld DIL device 200 for use with the above-described system 100 is illustrated. The exemplary handheld DIL device 200 includes a scissor-like actuating handle 202, a guide shaft 204, and an LED element 206. As illustrated, the scissor-like actuating handle 202 includes a pair of arms 208, which, when squeezed together, are configured to actuate the LED element 206 from a location within the guide shaft 204, to a location partially outside of a distal end 210 of the guide shaft 204. This actuation allows for the guide shaft 204 to be inserted into the nasal cavity of a subject, and the LED element 206 to then be actuated out of the distal end 210 for providing treatment within the nasal cavity 102.

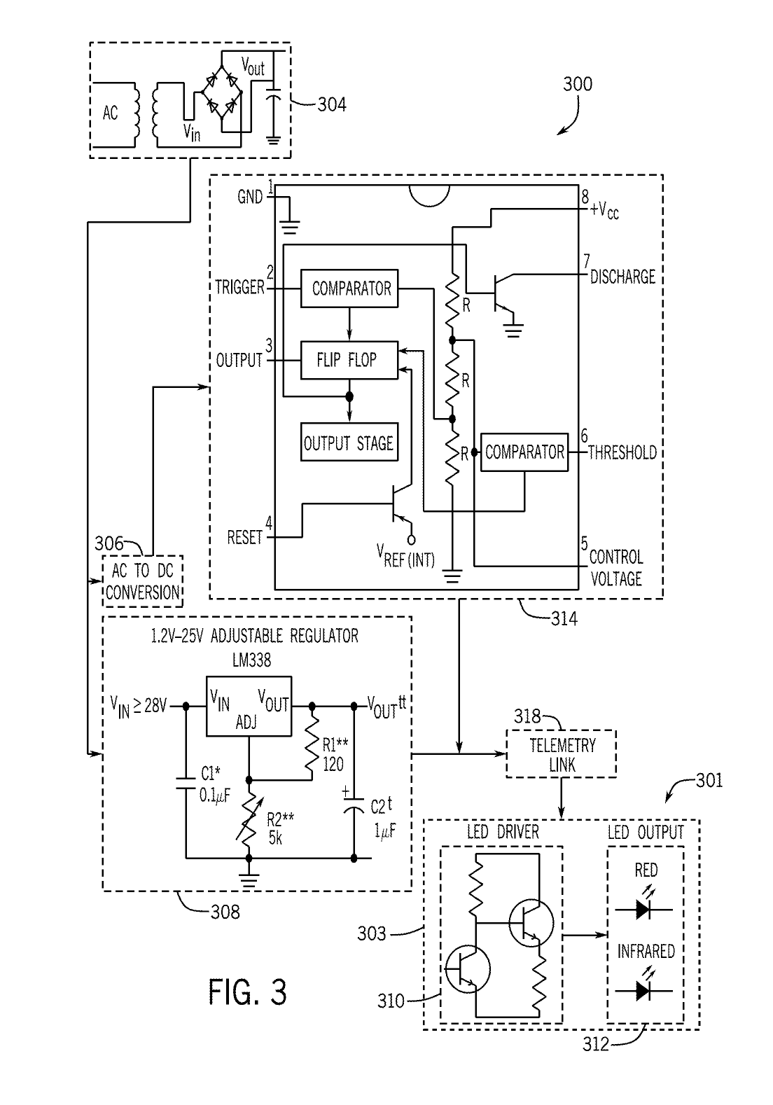

Referring now to FIG. 3, another example of a system 300 for DIL delivery is illustrated. The system 300 is designed to function with an implantable device 301 having a housing 303 sized to be located within the submucosa of the nasal cavity, in close proximity to the cribriform plate of the subject, and below a cranial base of the subject. The system 300 is substantially similar to the system 100, described above, and as such, similar features are labeled with similar number in the 300 series (e.g., power source 104 and power source 304, gain controller 108 and gain controller 308). Differences and similarities between the system 100 and the system 300 will be make clear in the following description. It will be appreciated, however, that features of the system 100 may be additionally added to the system 300, and vice versa, as desired for a given application. These combinations are contemplated herein and do not depart from the scope of the present disclosure.

Again, the system 300 can be designed to achieve direct access to the limbic system, and can provide therapy and/or treatment related to various disorders, such as, for some non-limiting examples, sexual dysfunction (e.g., decreased libido), depression anxiety, cognitive impairment, and the like.

The system 300 again includes a power source 304. Although the illustrated example includes an AC power source, in many instances the power source 304 can be a battery. The battery can be replaceable or rechargeable, as desired. Again, in the instances that a dedicated DC source is not included, such as is illustrated in FIG. 3, the system 300 may include a voltage regulator 306 that converts power into a lower DC source.

Further, in some instances, the power source 304 can comprise a remote generator, configured to wirelessly power the implantable device 301. In these instances, the remote generator can be a miniaturized generator disposed within a wearable article of clothing, such as, for example, a pair of wearable eye glasses.

Additionally, the system 300 again includes a gain controller 308 that allows the user to adjust the operational power and, for example, change light intensity of the various LEDs 312 of the implantable device 301. The implantable device 301 includes an LED driver 310 and the various LEDs 312.

The various components of the system 300 are functionally coupled to a controller 314, which is programmed to operate the system 300, for example, by controlling pulsing, frequencies, intensity modulation, and the like.

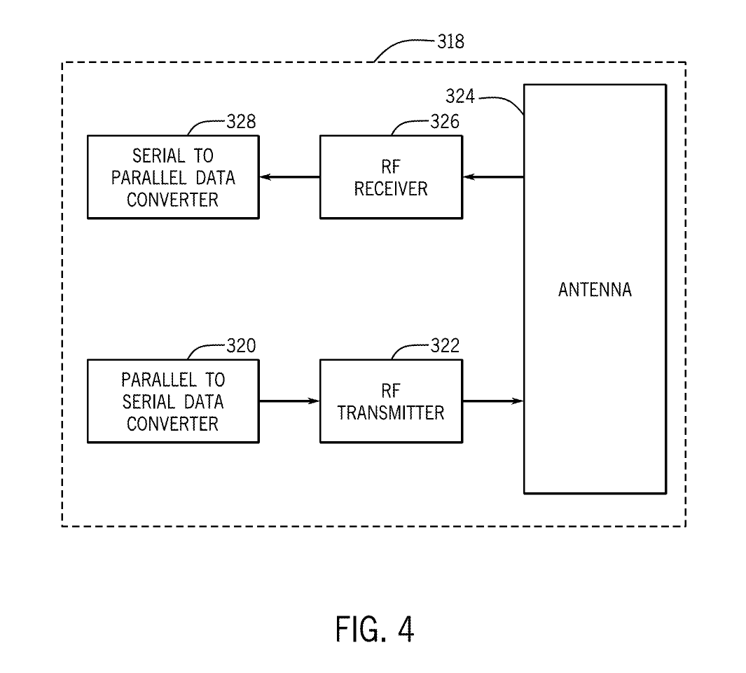

However, the implantable device 301 is wirelessly controlled by the controller 314, and as such, the system 300 further includes a telemetry link 318 to provide communication between the controller 314 and the implantable device 301. As shown in FIG. 4, the exemplary telemetry link 318 can include a parallel to serial data converter 320, an RF transmitter 322, an antenna 324, an RF receiver 326, and a serial to parallel data converter 328.

Referring now to FIG. 5, the implantable device 201 can be implanted within a nasal region 500 of a subject, within the submucosa or submucosal tissue 502 of the nasal cavity 102. The submucosal tissue 502 is disposed between the nasal cavity 102 and the septal cartilage 506 of the subject. In many instances, the implanted device 201 is implanted within the submucosal tissue 502 of the subject, in close proximity to the cribriform plate 120 (shown in FIG. 1). For example, in some instances, the implantable device 501 can be implanted between 1 mm and 4 cm from the cribriform plate 120.

Because of the location of the implantable device 301 within the submucosal tissue 502, the NIR light will be more efficiently delivered to the desired areas of the brain. Further, it allows for a more convenient therapy, which should lead to improved subject adherence. Additionally, by implanting the implantable device 301 within the submucosal tissue 502, it does not require neurosurgery and will be less likely to induce infections, abscesses, meningitis, intracranial hemorrhage, or CSF leaks. Furthermore, after placement of implantable device 301, frequent visits in specialty care will not be necessary, and subjects can return for follow up to their primary psychiatrist, with only periodic visits with the tertiary care DIL specialist psychiatrist.

The systems 100, 300 described herein can be used to administer near-infrared light and red light to regions of the cerebrum in a dosimetry and duration sufficient to treat a brain disorder. Near-infrared light having a wavelength of 750 nm to 1200 nm, together with red light having a wavelength of between 600 and 749 nm, can be administered from an apparatus configured to administer near-infrared light and red light through the nasal cavity 102 into one or more regions of the cerebrum including, but not limited to, the ventromedial prefrontal cortex (vmPFC), subgenual anterior cingulate cortex (ACC), or the olfactory bulb 118. In many cases, the near-infrared light and/or the red light may be administered through the cribriform plate 120, which allows for the light to be shed through holes or “windows” in the skull, with no bone interposed.

The DIL systems 100, 300 will be used in an outsubject specialty care setting. Outsubject psychiatrists will refer subjects with treatment-resistant depression to tertiary care centers, where a team of a psychiatrist and an ENT specialist will evaluate the appropriateness of the treatment for the subject, deliver initial treatment with a handheld DIL device, such as the exemplary handheld DIL device 200 (twice a week for 8 weeks, 10 min each application), and ultimately place an implantable DIL, such as the exemplary implantable device 301, for maintenance of subjects in whom treatment has proved effective, as measured by standardized rating scales for depression severity and functional status including the HAMD-17, IDS, MGH CPFQ, and Q-LES-Q.

A variety of disorders can be treated using the DIL systems and methods described herein. For example, the DIL systems and methods described herein can be used to treat a variety of brain disorders. The ability of the systems 100, 300 to administer light through the cribriform plate 120, allows for efficient targeting of affected brain structures (e.g., the olfactory bulb 118) that are common to such brain disorders, including depression and dementia.

Brain disorders that can be treated using the DIL systems and methods include, but are not limited to, depressive disorders, anxiety disorders, trauma- and stressor-related disorders, disorders manifesting with suicidal ideation or just suicidal ideation, alcohol use disorder, substance use disorder, sexual dysfunction disorders, neurocognitive disorders, attention deficit and hyperactivity disorder and other neurodevelopmental disorders, sleep-wake disorder, disorder associated with chronic fatigue syndrome, disorder associated with fibromyalgia, somatic symptom disorder, eating disorder, psychotic disorder, obsessive-compulsive disorder, cluster-B personality disorder or a disruptive, impulse-control, and conduct disorder and otorhinolaryngology disorders, treatment-resistance for any of the aforementioned conditions and disorders and for any of the indications listed elsewhere in this application.

Depressive disorders include, but are not limited to, unipolar and bipolar disorders, premenstrual dysphoric disorder and seasonal affective disorder, and complicated grief.

Anxiety disorders include, but are not limited to, generalized anxiety disorder, panic disorder, specific phobias, social anxiety disorder, separation anxiety, and agoraphobia.

Trauma- and stressor-related disorders include, but are not limited to, PTSD and complicated grief. In particular, acute treatment after trauma exposure can be administered to reduce or ameliorate PTSD, depression, and suicidal ideation. Treatment can further enhance cognitive and/or motor performance for situations requiring exceptional physical and/or mental demands (e.g., combat).

Sexual dysfunction disorders include, but are not limited to, decreased libido, anorgasmia, delayed ejaculation and erectile disorder, and medications' sexual side-effects.

Neurocognitive disorders include, but are not limited to, Alzheimer's disease, traumatic brain injury and dementia (e.g. frontotemporal dementia and related disorders), Parkinson's disease and other synucleinopathies, stroke-TIA prevention, amyotrophic lateral sclerosis, multiple sclerosis, headache, epilepsy, medications' cognitive side-effects, including side-effects from neuromodulation (e.g., electroconvulsive therapy).

Neurodevelopmental disorders include, but are not limited to, Down Syndrome, intellectual disabilities, learning disorders, language, reading, and speech disorders.

Sleep-wake disorders include, but are not limited to, insomnia disorder and restless leg syndrome.

Somatic symptom and related disorders include, but are not limited to, somatic symptom disorder, illness anxiety disorder and conversion disorder.

Eating disorders include, but are not limited to, bulimia nervosa, binge-eating disorder and obesity.

Psychotic disorders include, but are not limited to, negative symptoms of schizophrenia.

Obsessive-compulsive (OC) disorders include, but are not limited to, OC-related disorders according to DSM-5.

Cluster-B personality disorders include, but are not limited to, borderline personality disorder and antisocial personality disorder.

Disruptive, impulse-control, and conduct disorders include, but are not limited to, oppositional defiant disorder, intermittent explosive disorder, conduct disorder, antisocial personality disorder, pyromania and kleptomania.

Otorhinolaryngology disorders include, but are not limited to anosmia, chronic allergic rhinitis, chronic, and recurrent sinusitis; other ENT indications are prevention of post-operative complications and induction of post-operative wound healing, as well as treatment or prevention of diseases of the sinonasal mucosa.

Developmental brain disorders that can be treated using the DIL systems and methods described herein include, but are not limited to, autism spectrum disorder, Down syndrome, and ADHD.

Maternal psychiatric illnesses that occur during pregnancy and the post-partum period (including during breast-feeding) can also be treated using the DIL systems and methods described herein.

Disorders that affect other organs (i.e., organs other than the brain including those disposed within the head or those disposed elsewhere within the body) or other systems generally can also be treated using the DIL systems and methods disclosed herein. In these cases, DIL represents a port of entry for shedding light into, for example, the ocular structure, cerebral nerves, cerebrospinal fluid, the vascular system, and/or lymphatic system. By these or other means DIL affects distant targets in the body. For instance, DIL systemic antioxidant, anti-inflammatory, and pro-metabolic effects could be used for the stabilization of critical care subjects.

As such, the systems and methods disclosed herein can additionally be used to treat the following: immune and autoimmune disorders; obesity; metabolic syndromes including, but not limited to, hyperglycemia; hypometabolic syndromes, where energy supplementation might be indicated, including but not limited to disorders of alimentation, of gastrointestinal absorption, anorexia, and/or low Body Mass Index (BMI). The systems and methods disclosed herein can also be used to decrease cardiovascular risk (e.g., decrease the risk of myocardial infarction, ischemic stroke, and various other cardiovascular risks) by means of primary or secondary prevention. Additionally, the systems and methods disclosed herein can be used as an anti-aging aid and a means for rejuvenation of both the mind and body. Furthermore, the systems and methods disclosed herein can be used to re-establish or enhance sensitivity (e.g., responsiveness) to existing treatments for brain or other organs' disorders or for systemic disorders. These applications that are not limited to the brain or to the head are also exemplified by, but not limited to, the treatment and prevention of infections.

Disorders treated using the DIL systems and methods of the invention will decrease the symptoms associated with these disorders. As used herein “a decrease in symptoms” associated with a disorder refers to at least about 0.05 fold less symptoms (for example 0.1, 0.2, 0.3, 0.4, 0.5, 1, 5, 10, 25, 50, 100, 1000, 10,000-fold or more less) typically exhibited in a subject not undergoing DIL therapy or in a subject prior to undergoing DIL therapy according to the methods described herein. “Decreased” as it refers to “a decrease in symptoms” also means at least about 5% less symptoms (for example 5, 6, 7, 8, 9, 10, 15, 20, 25, 30, 35, 40, 45, 50, 55, 60, 65, 70, 75, 80, 85, 90, 95, 99 or 100% less) typically exhibited in a subject not undergoing DIL therapy or in a subject prior to undergoing DIL therapy according to the methods described herein. Amounts can be measured by clinicians according to methods known in the art for evaluating symptomatic subjects.

Methods for administering near-infrared light and red light comprise administering near-infrared light having a wavelength of 750 nm to 1200 nm, together with red light having a wavelength of between 600 and 749 nm.

In some aspects, the near-infrared light is administered at a wavelength of about 825 nm or about 850 nm or at a wavelength of about 830 nm to about 808 nm. Near-infrared light can comprise about 50% to about 75%, to about 99% of the total light administered.

In some other aspects, the red light is administered at a wavelength of about 633 nm or at a wavelength of about 620 nm to about 633 nm. Red light can comprise about 1% to about 50% of the total light administered.

Additionally, the near-infrared light and red light can be administered simultaneously, continuously, or in pulses (e.g., the near-infrared light and red light are administered together in a series of alternating pulses) from the system 100. For example, the near-infrared light can be administered at a wavelength of about 795 nm to about 830 nm and red light can be administered at a wavelength of about 650 nm to about 720 nm in a pulse that alternates with a next pulse, wherein near-infrared light is administered at a wavelength of about 721 nm to about 794 nm and red light is administered at a wavelength of about 600 nm to about 649 nm.

In yet some other aspects, the near-infrared light and red light are administered in a series of alternating pulses, wherein near-infrared light is administered at a wavelength of about 760 nm to about 830 nm and red light is administered at a wavelength of about 620 nm to about 680 nm.

The dosimetry of near-infrared light and red light administered to a human subject in need of treatment can comprise one or more of: between about 5 mW and 2 W power, between about 5 mW/cm² to about 700 mW/cm² irradiance, between about 1 J/cm² to about 300 J/cm² fluence, with continuous light or 1 Hz to about 100 Hz pulses of near-infrared light and red light. In specific embodiments, the irradiance is between about 22 mW/cm² to about 33 mW/cm² and the fluence is between about 9.56 J/cm² to about 12 J/cm². In other specific embodiments, the dosimetry of near-infrared light and red light administered to the human subject comprises between about 22 to about 33 mW/cm² irradiance, between about 9.56 to about 12 J/cm² fluence and about 10 Hz pulses of near-infrared light and red light.

Typically, the duration of administration of near-infrared light and red light is about 1 minute to about 120 minutes. The frequency of administration could be a single treatment or multiple treatments occurring once a day, as often as 20 times a day, or as little as once a week.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (as well as fractions thereof unless the context clearly dictates otherwise).

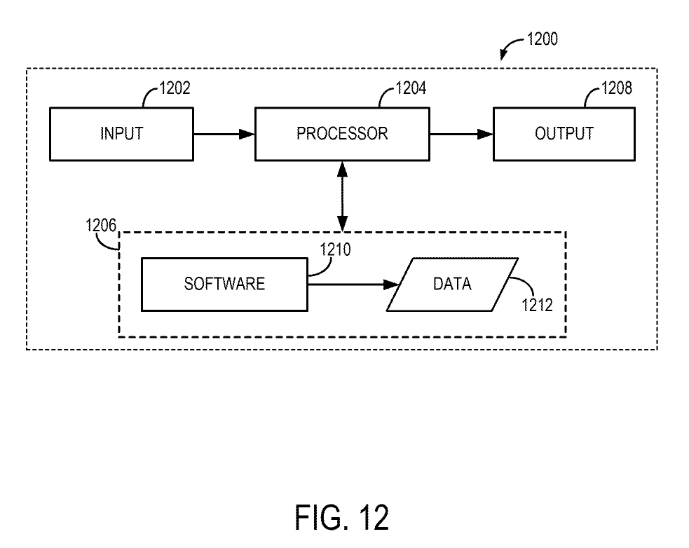

Referring now to FIG. 12, a block diagram of an example of a controller 1200 that can be integrated either of the systems 100, 300 to perform the methods described in the present disclosure is shown. Specifically, in some instances, the controller 1200 can replace either of the controllers 114, 314. The controller 1200 is generally implemented with a hardware processor 1204 and a memory 1206.

The controller 1200 generally includes an input 1202, at least one hardware processor 1204, a memory 1206, and an output 1208. The controller 1200 can also include any suitable device for reading computer-readable storage media. The controller 1200 may be implemented, in some examples, by a workstation, a notebook computer, a tablet device, a mobile device, a multimedia device, a network server, a mainframe, one or more controllers, one or more microcontrollers, or any other general-purpose or application-specific computing device. The controller 1200 may operate autonomously or semi-autonomously, or may read executable software instructions from the memory 1206 or a computer-readable medium (e.g., a hard drive, a CD-ROM, flash memory), or may receive instructions via the input 1202 from a user, or any another source logically connected to a computer or device, such as another networked computer or server.

In general, the controller 1200 is programmed or otherwise configured to implement the methods and algorithms described above. For instance, the controller 1200 is programmed to provide power to either of the handheld device 200 or the implantable device 301 to cause either device to emit the near-infrared light and/or the red light, in accordance with any of the dosimetries, durations, or pulse sequences described herein.

The input 1202 may take any suitable shape or form, as desired, for operation of the controller 1200, including the ability for selecting, entering, or otherwise specifying parameters consistent with performing tasks, processing data, or operating the controller 1200. In some aspects, the input 1202 may be configured to receive data, such as data acquired through a user interface. Such data may be processed as described above to determine the correct dosimetry, duration, pulse sequence, or any other testing variable.

The memory 1206 may contain software 1210 and data 1212, such as data acquired with a user interface, and may be configured for storage and retrieval of processed information, instructions, and data to be processed by the one or more hardware processors 1204. In some aspects, the software 1210 may contain instructions directed to emit the near-infrared light and/or the red light at various organs and/or systems within the head or other parts of the subject, as desired.

The present invention is additionally described by way of the following illustrative, non-limiting Examples that provide a better understanding of the present invention and of its many advantages.

### EXAMPLES

The following Examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following Examples do not in any way limit the invention.

### Example 1. Computational Modeling of Optimal DIL Parameters

While transcranial light reaches the dorsolateral prefrontal cortex (dlPFC), intranasal light is expected to be ideal to shed light on the ventromedial prefrontal cortex (vmPFC). A computerized model was used to test the expected penetration of DIL light and verified that indeed the light penetrates sufficiently to produce an antidepressant effect deposition or energy density.

The model used for the simulation was based on the Monte Carlo algorithm, which is very accurate and generally has been used as the gold-standard in most optical imaging system evaluations. The assumptions for the model were that: 1. an adult brain atlas (colin27) is representative of the adult brain anatomy in general population; and 2. the optical properties for the different tissues, as described in the literature, are close to the real optical properties of individual subjects.

The computational modeling was used to assess optimal DIL parameters in order to maximize light penetration to the brain target areas for the treatment of disorders including MDD. Based on maximum benefits reported in preclinical animal research, a NIR fluence of about 0.5-2 J/cm² at the olfactory bulb, ventromedial prefrontal cortex, and subgenual anterior cingulate, was administered, while shedding no more than 5 J/cm² of NIR in any other brain area.

The desired fluence was reached in target brain areas by shedding light at 850 nm wavelength, 100 mW/cm² irradiance, 10 Hz pulse rate, 50% duty cycle, 10 minute exposure.

Computerized simulations of the penetration of near-infrared (NIR) and red light into the brain from a light source located deeply in the nasal cavity was performed. Coefficients of light penetration, refraction and reflection were attributed to the anatomical parts lying between the light source and the brain target areas. These coefficients of penetration were based on the tissue composition of each anatomical area (e.g. degree of vascularization). Optical parameters related to the light permeability at different wavelengths are available for human live tissues. Once these wavelength-specific, anatomical maps of light permeability were completed, the expected fluence (at the level of brain targets) was assessed, through repeated computational simulations based on variations of isolated parameters, including wavelength (600-1040 nm), pulse vs. continuous light, frequency of pulses (1-100 Hz), irradiance (5-700 mW/cm²), exposure time (1-15 min) and depth of light source in the nasal cavity (from nostril to the vicinity of cribriform plate in submucosal space).

By using the computerized modeling described above, we tested the penetration of light with set parameters, corresponding to the specifications of DIL (deep intranasal), and its deposition at the target brain area, the vmPFC.

The penetration of light from a superficial source was also modeled and tested for the energy deposition on vmPFC.

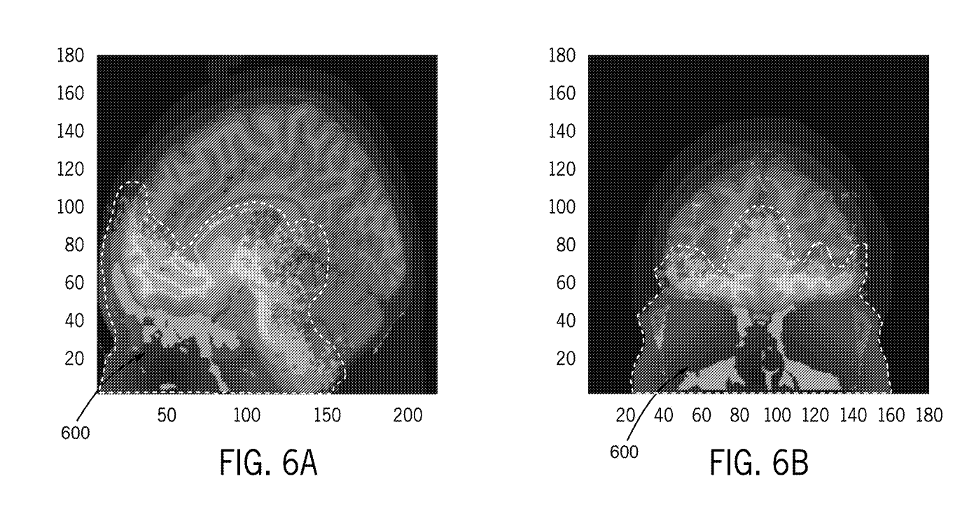

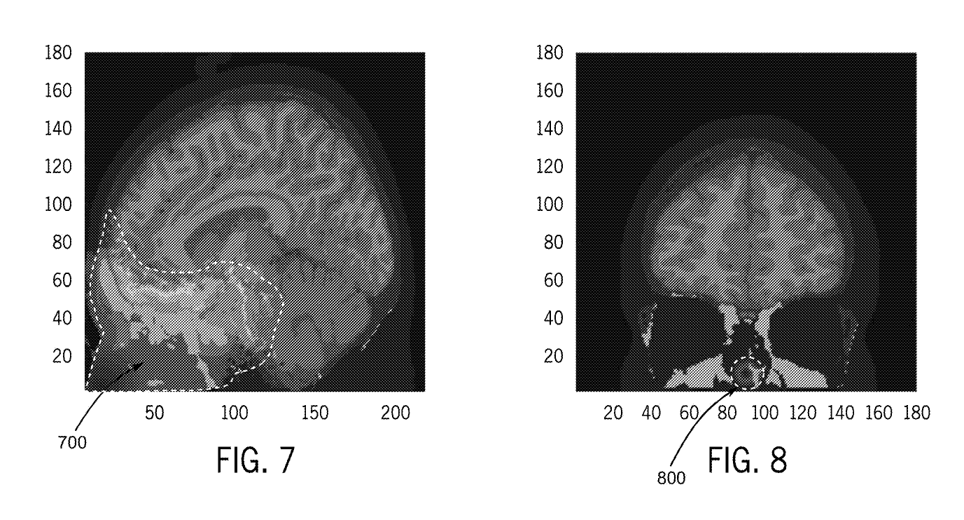

Assuming equal power and exposure, DIL produced at least more than twice (ratio 2.2) as much light deposition 600 on vmPFC (shown in FIGS. 6A and 6B), compared to light deposition 700 provided by the superficial source (shown in FIG. 7), when the DIL light source 800 was positioned central to the nasal cavity (shown in FIG. 8).

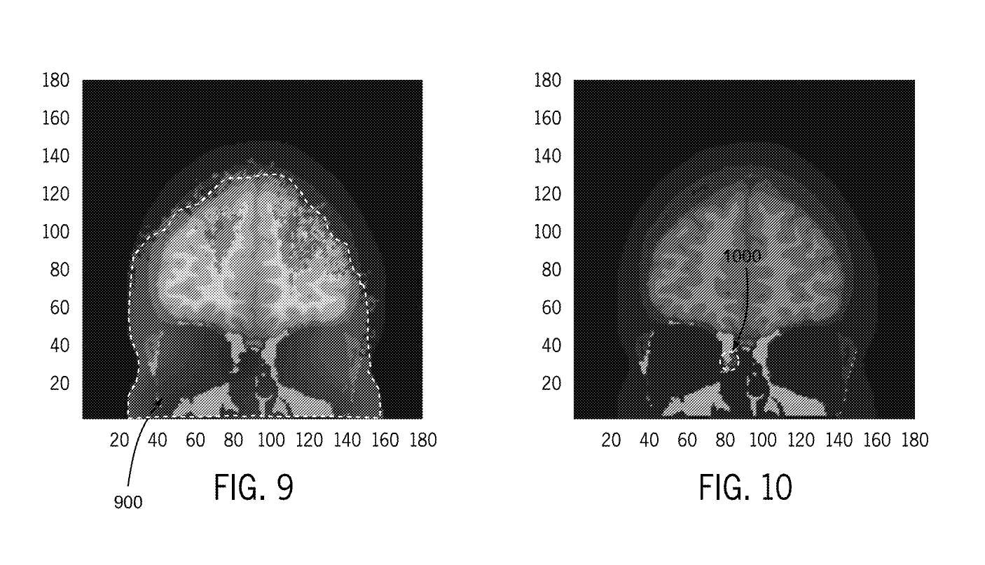

Even greater light deposition 900 into the vmPFC (shown in FIG. 9) was obtained when the DIL light source 1000 was positioned in the upper portions of the nose (shown in FIG. 10). The latter source position for DIL resulted in a robust energy deposition of 0.12-0.11 J/cm³ and 1.2-1.1 J/cm³ for a DIL power source of 100 mW and 1 W for 10 min, respectively. A typical source of light placed in the nostril (e.g. superficial source at 25 mW for 20 min 50% duty cycle) resulted in a negligible energy deposition of 0.12*10−6 J/cm³ on vmPFC, which is 1 million times less than the light shed by DIL.

In sum, our model demonstrated the advantage of DIL over superficial placements in the nostril of NIR light in order to shed light with antidepressant effect in the key target regions of vmPFC.

### Example 2. DIL Antidepressant Efficacy

Next, the antidepressant effect of DIL in subjects suffering from MDD resistant to treatment will be assessed.

Clinical Trial Design: A pilot study on the use of DIL (handheld) as a treatment for depressive symptoms in 10 subjects with MDD (diagnosed by SCID).

Inclusion Criteria: Age: 18-65; women and men; baseline Hamilton Rating Scale for Depression (HAM-D-17)≥16; resistant to at least three adequate antidepressant treatments (ATRQ); all women of reproductive age will be using adequate birth control. Subjects currently on an antidepressant will need to be on a stable dose for at least six weeks.

Exclusion Criteria: Pregnancy or lactation; specific psychotherapies for depression started in the last 8 weeks; history of device-based treatments for MDD; substance dependence or abuse active in the past 6 months; any psychotic disorder or psychotic episode; bipolar disorder; unstable medical illness; active suicidal or homicidal ideation (C-SSRS>3); implants in the head; use of light-activated drugs; implanted metal devices in the body. Other exclusion criteria should include subjects with aberrant intranasal anatomy including deviated septum or chronic rhinosinusitis.

Scales: After written consent, subjects will undergo SCID-I/P for the diagnosis of MDD. The HAM-D-17 for baseline severity assessment of depression; the Inventory for Depressive Symptomatology (IDS) and the Clinical Global Impression (CGI) will be used to track depressive symptoms at weekly study visits for 8 weeks.

Treatment: All 10 subjects will undergo regular sessions with the DIL handheld device, however half will be randomly assigned to receive sham sessions and will be the study controls. Treatment will be intranasal using a DIL prototype of handheld device; NIR light parameters are 850 nm, 100 mW/cm², 10 Hz, 50% cycle for 10 minutes per session. Changes to the parameters will be made to optimize penetration-based on findings from part-A-prior to initiation of the pilot trial. The DIL technique involves the insertion in the subject nasal cavity of the handheld DIL lead (tip in proximity of the cribriform plate) by ENT under direct endoscopic visualization for 10 minutes. Dose will change to 15 minutes and 20 minutes (and to lower dose of 5 min), as tolerated, if no response at 4 weeks. The treatment will be administered twice a week for 8 weeks, for a total of 16 sessions (MGH DCRP).

Brain Imaging (fcMRI): all subjects will undergo three functional connection MRI (fcMRI) scans: prior to treatment and at week 4 and week 8 (after study completion). Subjects will undergo a 3 Tesla structural and functional MRI in a Siemens Trio (Siemens, Elrangen, Germany). Protocols have been developed and validated at the MGH Martinos Center to assess connectivity of different brain areas at rest and under emotional stimuli.

Safety, tolerability will be monitored by an ENT and by a psychiatrist.

The Primary Outcome Measure will be collected through treatment-blind phone assessments of depression (baseline and every 2 weeks) and through self-rated assessments.

### Example 3: Transcranial Photobiomodulation for the Treatment of MDD

The following example is provided as further evidence for the efficacy of photobiomodulation with NIR and red light as a treatment for MDD. Although the NIR and red light were not delivered using a DIL system, as illustrated in Example 1 above, the DIL systems and methods described herein provide significantly higher penetration of the NIR and red light into the cerebrum of the subject when compared to a superficial source, and as such should be even more efficacious than the following transcranial photobiomodulation example.

Inclusion and Exclusion Criteria:

Adult subjects (age 18-65 years) meeting the (DSM-IV SCID) criteria for MDD, with at least a moderate degree of depression severity (Hamilton Depression Rating Scale, HAM-D₁₇ total score ranging 14-24), were included in the study after providing written informed consent. The MGH IRB required a maximum permitted HAM-D₁₇ score of 24 to prevent inclusion of subjects at greater risk of suicide. During the current episode, subjects could have failed no more than one FDA-approved antidepressant medication (for at least 6 weeks) and no more than one course of structured psychotherapy for depression (for at least 8 weeks). Other exclusionary conditions included active substance use disorders (prior 6 months), lifetime psychotic episodes, bipolar disorder, active suicidal ideation and homicidal ideation, in addition to unstable medical illness and recent stroke (prior 3 months). Women of child-bearing potential were required to use a birth-control method if sexually active; pregnancy and lactation were exclusionary. To allow maximum light penetration and to minimize potential risks of local tissue damage from the use of NIR, the following conditions were also exclusionary: 1. having a forehead skin condition; 2. taking a light-activated medication (prior 14 days); and 3. having a head-implant.

Study Design and Treatment:

Eligible subjects were randomized to an 8-week study with, twice weekly, double-blind t-PBM NIR vs. sham. At each treatment session, NIR or sham were administered to the forehead bilaterally (Omnilux New U, light emitting diode, manufactured by Photomedex Inc.). The device used for this study emitted NIR at a wavelength of 830 nm, corresponding to the peak absorption spectrum for our biological target: cytochrome-C oxidase. In cadaver heads, the same device delivered 2% of the light at a penetration depth of 1 cm from the skin surface on frontal areas. A 2% penetration rate allows a NIR energy density equivalent to the fluence inducing neurological benefit in animal models [fluence: 0.85-1.27 J/cm², not accounting for blood related attenuation of light on the prefrontal cortex (i.e., optical energy per unit area, expressed in joules per cm²)]. As we were targeting the dorsolateral prefrontal cortex (dlPFC), we directed the NIR to the F3 (left) and F4 (right) sites on the forehead-derived from the EEG placement map.

The course of t-PBM was 8 weeks with a total of sixteen sessions; twice a week sessions had been acceptable and well-tolerated in our proof of concept study. The study clinician had the option to adjust the duration of light exposure after completion of week 3 and week 5 (after 6 and 10 sessions respectively) from 20 minutes to 25 and 30 minutes, respectively. Instructions were to increase exposure per protocol, as tolerated, to maximize the antidepressant effect. The exposure time was designed to allow a fluence of 60 J/cm², despite relatively low power density (irradiance) of 33.2 mW/cm², based on settings reported by the manufacturer. Similar and greater NIR fluences have been associated with antidepressant response and improved cognition in prior reports. All but three subjects remained on stable antidepressant treatment during the trial; their data were censored after change in concomitant psychoactive therapies.

Randomization and Blinding:

Two t-PBM device types were available for each modality (NIR and sham). The apparent behavior of the devices was identical for both modalities. However, only NIR-mode t-PBM device produced the therapeutic NIR energy. NIR light is invisible and undetectable to subjects and physicians. The study research assistant used permuted block randomization with varying block sizes to randomize subjects in 1:1 fashion to each pair of instruments as “A” and “B”. Only the research assistant was able to identify each pair of instruments as “A” and “B”. The investigators and the subjects remained blind to the subject assignment, since the label on each device was covered prior to treatment administration. Photomedex, Inc. provided the blinding codes of NIR and sham for each labeled pair of devices, which were kept in a sealed envelope at the study site.

Clinical Outcome Measures:

The primary outcome measure was the total score of the HAM-D₁₇ for depressive symptoms, in accordance to our initial report prior to study enrollment (Clinicaltrials.gov).

Analyses:

The study hypothesis that t-PBM NIR-mode will decrease HAM-D₁₇ scores in study subjects significantly more than the sham was tested. The dependent variable was the primary outcome of depression severity (as measured by the HAM-D₁₇ total score); the independent variable was the comparison between the NIR and sham groups. An intent-to-treat approach was used with last observation carried forward (LOCF) and a Mann-Whitney U test, comparing the change in the total severity score from baseline to endpoint. All analyses were repeated in completers (n=13). The self-rated QIDS total score for depression (LOCF and completers analyses) were examined post-hoc. Rates of antidepressant response and remission at endpoint for the two groups were also compared. Rates of antidepressant response and remission were calculated according to the HAM-D₁₇ total score (≥50% decrease and score ≤7, respectively) and the CGI-Improvement scale (response equal to score 1 or 2).

All response and remission rates were compared by Pearson's Chi-square test. To calculate the effect-size of t-PBM, the Cohen's d formula for the change of HAM-D₁₇ total score from baseline to endpoint was adopted. For any type of adverse event, its frequency was reported and its characteristics, relation to the treatment, any action taken, and final outcome were described. Baseline characteristics for the two groups were compared by Mann-Whitney U test and Pearson's Chi-square test, respectively for continuous and nominal variables. For all analyses significance was set at p≤0.05.

Results:

There were no significant differences among the two groups at baseline in terms of demographic and clinical characteristics as well as concurrent antidepressant treatment, except for a history of more MDD episodes in the t-PBM NIR group (mean 4.3±1.7 vs. 2.6±1.8; z=1.988; p=0.047). Roughly half of the sample in the NIR-mode (40%; n=4) and in the sham-mode (64%; n=7) groups had not received an antidepressant medication or psychotherapy during the current MDD episode. Three subjects per group had tried psychotherapy during the current episode. Three NIR and two sham subjects had tried one antidepressant medication during the current episode. Two and one subjects in the NIR and sham group, respectively, had undergone two medication trials. During the study, all subjects continued their baseline antidepressant treatment, if any, except one subject who discontinued their psychotherapy at baseline.

Antidepressant Effect:

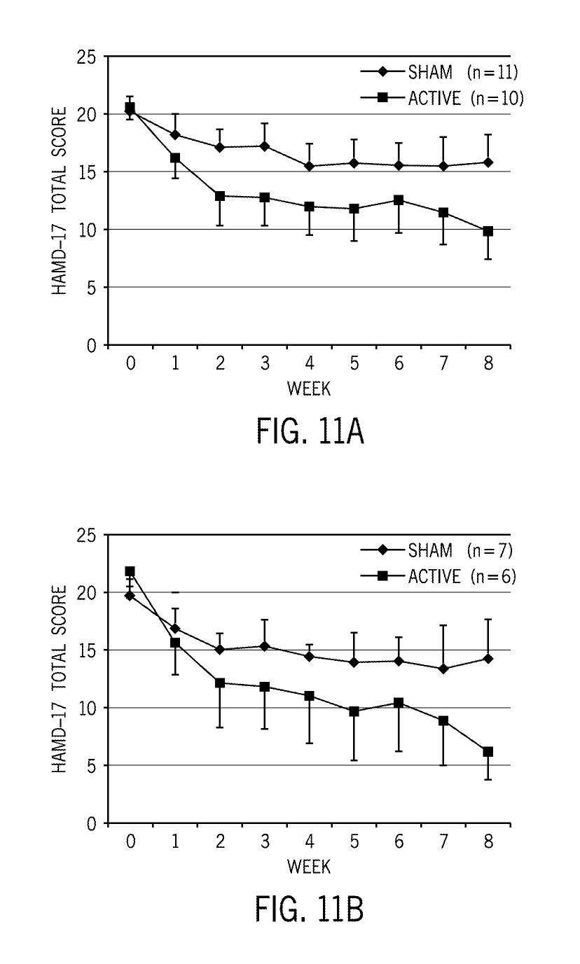

At endpoint, the mean change in HAM-D₁₇ total score in subjects receiving t-PBM in NIR-mode (n=10) was significantly greater than in subjects receiving sham-mode (n=11): −10.8±7.55 vs. −4.4±6.65 (LOCF, z=1.982, p=0.047). Among completers, the mean change in HAM-D₁₇ total score in subjects receiving t-PBM in NIR-mode (n=6) was also significantly greater than in subjects receiving sham-mode (n=7): −15.7±4.41 vs. −6.1±7.86 (z=2.158, p=0.031). FIGS. 11A and 11B illustrate the mean HAM-D₁₇ total scores over the course of the study for the two t-PBM groups.

The effect-size for the antidepressant effect of t-PBM, based on change in HAM-D₁₇ total score at endpoint, was 0.90 (Cohen's d). At endpoint, response and remission per the HAM-D₁₇ occurred in 5 out of 10 (50%) subjects in the NIR-mode. In the sham-mode, response and remission occurred in 3 and 2 subjects out of 11, respectively (27% and 18%) (response: χ²=1.15; df=1; p=0.284; remission: χ²=2.39; df=1; p=0.122). Response in the NIR-mode was attained after 2 weeks of t-PBM (n=3) and after 3 and 4 weeks (n=1 for each time point). Response in the sham-mode occurred after 3, 4 and 5 weeks of t-PBM (n=1 for each time point). At endpoint, 67% of NIR vs. 22% of sham subjects were at least “much improved” according to the CGI (χ²=3.88; df=1; p=0.049). In the post-hoc analyses, the antidepressant effect of t-PBM NIR-mode, measured by self-rated QIDS total scores, approached significance only in completers (Total sample: LOCF; n=20; −5.3±5.81 vs. −3.0±3.00; z=0.877, p=0.380. Completers: n=12; −9.8±4.09 vs. −4.3±3.04; z=1.874, p=0.061).

Blinding of Subjects and Clinicians:

None of the subjects reported excessive skin warming, which supported the blinding. All correlations between treatment assignment and its guess from the subjects were non-significant, with a 60% rate of correct guesses at week 4 (n=15; χ²=1.03; df=1; p=0.310) and 54% at week 8 (n=11; χ²=0.24; df=1; p=0.621). However, clinicians' guesses were significantly different among the two groups at both week 4 (n=14: χ²=4.66; df=1; p=0.031) and week 8 (n=10; χ²=4.28; df=1; p=0.038), with a 79% and 80% rate of correct guesses, respectively.

Discussion:

This study demonstrated a significant antidepressant effect of t-PBM NIR over sham. t-PBM was fairly well tolerated with none of the adverse events causing study discontinuation and only one case requiring dose adjustment. Attrition rates were the average for clinical trials.

The results are consistent with open-label reports that also demonstrated an antidepressant effect for t-PBM in MDD subjects and with a sham-controlled study on enhancement of attention bias modification for depression with t-PBM. The detection of a large effect-size of t-PBM (0.90) in MDD is also noteworthy, however common for small studies. The post-hoc analyses of the self-report measure of the antidepressant effect, while not reaching statistical significance in a smaller sample size, showed similar trends in terms of effect-size and p-value (p=0.06 in completers), despite the prediction of t-PBM assignment by subjects did not exceed chance (50%).

The present invention has been described in terms of one or more preferred embodiments, and it should be appreciated that many equivalents, alternatives, variations, and modifications, aside from those expressly stated, are possible and within the scope of the invention.

1. A method of controlling a device configured for treating a disorder of a subject, the method comprising:
providing power to an implantable device configured to be located within a submucosa of a nasal cavity of the subject to cause the implantable device to emit near-infrared light and red light directed to at least one of regions of an ocular structure, regions of a cerebrum, cerebral nerves, and cerebrospinal fluid, regions of a vascular system, and regions of a lymphatic system of the subject, the near-infrared light having a wavelength of 750 nm to 1200 nm, the red light having a wavelength of between 600 nm and 749 nm; and
wherein the device is configured to be implanted in a position to deliver the near-infrared light and the red light to the at least one of the regions of the ocular structure, the regions of the cerebrum, cerebral nerves, and cerebrospinal fluid, regions of the vascular system, and the regions of the lymphatic system in a dosimetry and duration sufficient to treat the disorder.

2. The method of claim 1, wherein the device is configured to deliver the near-infrared light and the red light to the regions of the cerebrum through at least a portion of a cribriform plate of the subject.

3. The method of claim 2, wherein the implantable device is sized to be located within the submucosa of the nasal cavity, in close proximity to the cribriform plate of the subject, and below a cranial base of the subject.

4. The method of claim 3, wherein the implantable device is sized to be located between 0.1 cm and 4 cm from the cribriform plate of the subject.

5. The method of claim 1, wherein providing power to the implantable device includes wirelessly delivering power to the implantable device using a remote generator.

6. The method of claim 1, wherein providing power to the implantable device includes delivering the power wirelessly form a wearable remote generator configured to be worn by the subject.

7. The method of claim 1, further comprising controlling the implantable device to deliver the near-infrared light and the red light simultaneously.

8. The method of claim 1, further comprising delivering the near-infrared light at one of a wavelength of about 825 nm, a wavelength of about 850 nm, or a wavelength of about 808 nm to about 830 nm.

9. The method of claim 1, further comprising delivering the red light at one of a wavelength of about 620 nm to about 633 nm or a wavelength of about 633 nm.

10. The method of claim 1, wherein the red light comprises about 1% to about 50% of a total light delivered.

11. The method of claim 1, further comprising controlling the implantable device to deliver the near-infrared light and red light together in a series of alternating pulses, wherein near-infrared light is at a wavelength of about 795 nm to about 830 nm and red light is at a wavelength of about 650 nm to about 720 nm in a pulse that alternates with a next pulse, wherein near-infrared light is at a wavelength of about 721 nm to about 794 nm and red light is at a wavelength of about 600 nm to about 649 nm.

12. The method of claim 1, further comprising controlling the implantable device to deliver the near-infrared light and red light in a series of alternating pulses, wherein near-infrared light is at a wavelength of about 760 nm to about 830 nm and red light is at a wavelength of about 620 nm to about 680 nm.

13. The method of claim 1, further comprising controlling the implantable device to deliver a duration of administration of near-infrared light and red light of about 1 minute to about 120 minutes per day.

14. The method of claim 1, further comprising controlling the implantable device to deliver a duration of administration of near-infrared light and red light of about 1 minute to 120 minutes once, twice or three times per week or daily or 20 times per day.

15. The method of claim 1, wherein the regions of the cerebrum are at least one of the ventromedial prefrontal cortex (vmPFC), subgenual anterior cingulate cortex (ACC) and the olfactory bulb.

16. The method of claim 1, wherein the disorder is at least one of a depressive disorder, an anxiety disorder, a trauma- and stressor-related disorder, a disorder manifesting with suicidal ideation or just suicidal ideation, a nicotine addiction disorder, an alcohol use disorder, a substance use disorder, a sexual dysfunction disorder, a neurocognitive disorder, an attention deficit and hyperactivity disorder, a sleep-wake disorder, a disorder associated with chronic fatigue syndrome, a disorder associated with fibromyalgia, a somatic symptom disorder, an eating disorder, a psychotic disorder, an obsessive-compulsive disorder, a cluster-B personality disorder, a disruptive, impulse-control, and conduct disorder, and an otorhinolaryngology disorder.

17. The method of claim 1, wherein the subject has been diagnosed with treatment resistant depression.

18. The method of claim 1, wherein near-infrared light comprises about 50% to about 99% of a total light delivered.

19. The method of claim 18, wherein near-infrared light comprises about 75% of the total light delivered.

20. The method of claim 1, wherein controlling the implantable device includes causing the implantable device to deliver the near-infrared light and red light to achieve between about 5 mW/cm² to about 700 mW/cm² irradiance, between about 1 J/cm² to about 300 J/cm² fluence, with one of continuous light and 1 Hz to about 100 Hz pulses of near-infrared light and red light.

21. The method of claim 20, wherein the irradiance is between about 22 mW/cm² to about 33 mW/cm², the fluence is between about 9.56 J/cm² to about 12 J/cm², and the dosimetry of near-infrared light and red light delivered to the subject comprises about 10 Hz pulses of near-infrared light and red light.

22. A device configured for treating a disorder of a subject, the device comprising:
a power source;
a light source configured to receive power from the power source to cause the light source to emit near-infrared light and red light directed, wherein the near-infrared light has a wavelength of 750 nm to 1200 nm and the red light has a wavelength of between 600 nm and 749 nm; and
a housing configured to be located within a submucosa of a nasal cavity of the subject to position the light source to deliver the near-infrared light and the red light toward at least one of regions of an ocular structure, regions of a cerebrum, cerebral nerves, and cerebrospinal fluid, regions of a vascular system, and regions of a lymphatic system of the subject in a dosimetry and duration sufficient to treat the disorder.

23. The system of claim 22, wherein the light source is configured to deliver the near-infrared light and the red light to the regions of the cerebrum through at least a portion of a cribriform plate of the subject.

24. The system of claim 22, wherein the housing is sized to be located within the submucosa of the nasal cavity, in close proximity to the cribriform plate of the subject, and below a cranial base of the subject.

25. The system of claim 22, wherein the power source is configured to wirelessly deliver the power to the light source.
